(19) United States
(12) Patent Application Publication
(10) Pub. No.: US 2025/0339029 A1
(43) Pub. Date: Nov. 6, 2025

(54) UNIVERSAL MEDICAL SYSTEM

(71) Applicant: Masimo Corporation, Irvine, CA (US)

(72) Inventor: Massi Joe E. Kiani, Laguna Niguel, CA (US)

(21) Appl. No.: 19/205,530

(22) Filed: May 12, 2025

Related U.S. Application Data
(63) Continuation of application No. 17/934,165, filed on Sep. 21, 2022, now Pat. No. 12,310,695.
Application No. 17/934,165 is a continuation of application No. 15/423,498, filed on Feb. 2, 2017, now Pat. No. 11,452,449.
Application No. 15/423,498 is a division of application No. 14/067,728, filed on Oct. 30, 2013, now Pat. No. 9,560,996.
(60) Provisional application No. 61/720,302, filed on Oct. 30, 2012.

(51) Int. Cl. A61B 5/00; A61B 5/0205; A61B 5/1455; G16H 40/40; G16H 40/63; G16H 40/67

(52) U.S. Cl. CPC A61B 5/002; A61B 5/0022; A61B 5/14551; A61B 5/14552; A61B 5/742; G16H 40/40; G16H 40/63; G16H 40/67; A61B 5/0205; A61B 2560/0266

(57) ABSTRACT

A system for operating third party proprietary software on a medical monitoring device operating native proprietary software and a system for obtaining compatible third party proprietary software for operation on the monitoring device.

### CROSS-REFERENCE TO RELATED APPLICATIONS

The present application is a continuation of U.S. patent application Ser. No. 17/934,165, filed Sep. 21, 2022 entitled “Universal Medical System,” which is continuation of U.S. patent application Ser. No. 15/423,498, filed Feb. 2, 2017, entitled “Universal Medical System,” which is a divisional of U.S. patent application Ser. No. 14/067,728, filed Oct. 30, 2013, entitled “Universal Medical System,” now U.S. Pat. No. 9,560,996, which claims priority benefit under 35 U.S.C. § 119(e) to U.S. Provisional Application Ser. No. 61/720,302, filed Oct. 30, 2012, entitled “Universal Medical Communication System,” the entirety of which is incorporated by reference herein.

The present application is also related to and incorporates by reference the subject matter disclosed in U.S. patent application Ser. No. 13/651,167, entitled Medical Monitoring Hub, filed Oct. 12, 2012 and U.S. patent application Ser. No. 12/723,526, entitled Open Architecture Medical Communication System, filed Mar. 12, 2012.

### FIELD OF THE DISCLOSURE

The present disclosure provides communications and display information for patient care equipment. In certain embodiments, the disclosure relates to providing an open architecture and cross platform communication, processing and display system.

### BACKGROUND

Hospitals, nursing homes, and other patient care facilities typically include patient monitoring devices and other patient care equipment at one or more bedsides in the facility. Patient monitoring devices, for example, generally include sensors, processing equipment, and displays for obtaining and analyzing a medical patient's physiological parameters. Physiological parameters include, for example, respiratory rate, blood gas levels, pulse, ECG, EEG, glucose and blood pressure, among others. Clinicians, including doctors, nurses, and certain other medical personnel use the physiological parameters obtained from the medical patient to diagnose illnesses and to prescribe treatments. Clinicians also use the physiological parameters to monitor a patient during various clinical situations to determine whether to increase the level of medical care given to the patient. Some patient care devices include proprietary software with proprietary indications that can only be used with their devices. Thus, often, multiple different redundant monitors may be required based on which software or indications a physician requires. Other patient care equipment can also used to assist in the care of the patient including medicine dispensing equipment, communication equipment, alarm signals and other devices.

Patient monitoring devices must be highly robust and able to tolerate component and device failures. Robustness is of particular importance where devices are used to monitor patient status in health care facilities. For example, if a component of a monitor fails, such as an alarm, the alarm conditions may go unnoticed. In some situations, if a patient monitoring device experiences a failure during operation, the failure may necessitate disabling the entire device.

### SUMMARY

An aspect of the disclosure includes the ability for a patient care device to display proprietary third party parameters and displayed information or display configurations in a safe and effective manner. For example, a care provider may prefer the accuracy of one patient care device with the display layout or derived parameter information of another system. A care provider can select on a patient care device the option to use third party proprietary displays and derived parameters. In an embodiment, the third party parameter can be selected and implemented based on a single use or use per patient. In other embodiments, the third party parameter can be selected and associated with a particular device.

Other aspects and embodiments of the disclosure are disclosed throughout this specification and in the drawings.

### BRIEF DESCRIPTION OF THE DRAWINGS

FIGS. 1A and 1B illustrate embodiments of a patient care system using a virtual machine.

FIGS. 1C and 2 illustrate examples of patient care networks.

FIG. 3 illustrates a typical pulse oximeter with display screen.

FIG. 4 illustrates an embodiment of a software application download screen.

### DETAILED DESCRIPTION

Various embodiments according to the disclosure will be described hereinafter with reference to the accompanying drawings. These embodiments are illustrated and described by example only, and are not intended to limit the scope of the disclosure.

Medical monitoring devices often include software for providing proprietary parameters or display configurations. For example, some medical monitoring devices include a proprietary wellness indication or proprietary parameters that are exclusive to a particular medical monitoring device. Medical professionals often have preferences regarding medical monitoring devices. For example, some medical professionals may prefer the accuracy of a particular medical monitoring device, but the display layout or proprietary parameters of other monitoring device. Due to the proprietary nature of this software, medical professionals are often required to make compromising choices between medical devices. In some cases, medical professionals use redundant devices.

The present disclosure provides solutions to this problem by providing a system to allow third party proprietary software to operate on any medical monitoring device. This allows a medical professional to pick and choose a monitoring device based on core functionality without compromising on desired third party proprietary parameters or display and alarm settings.

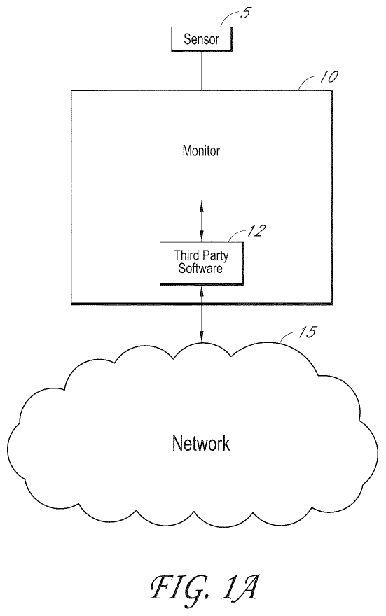

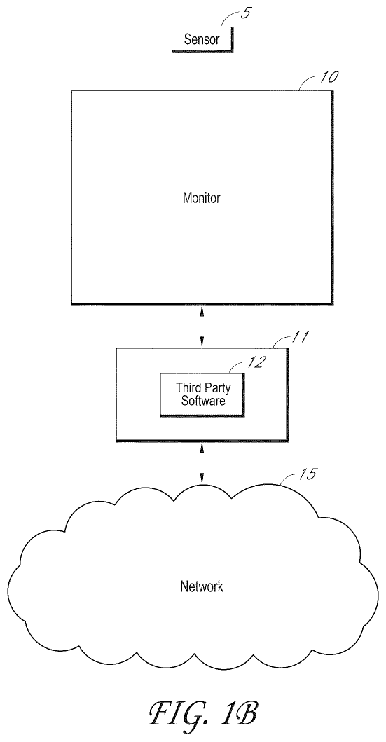

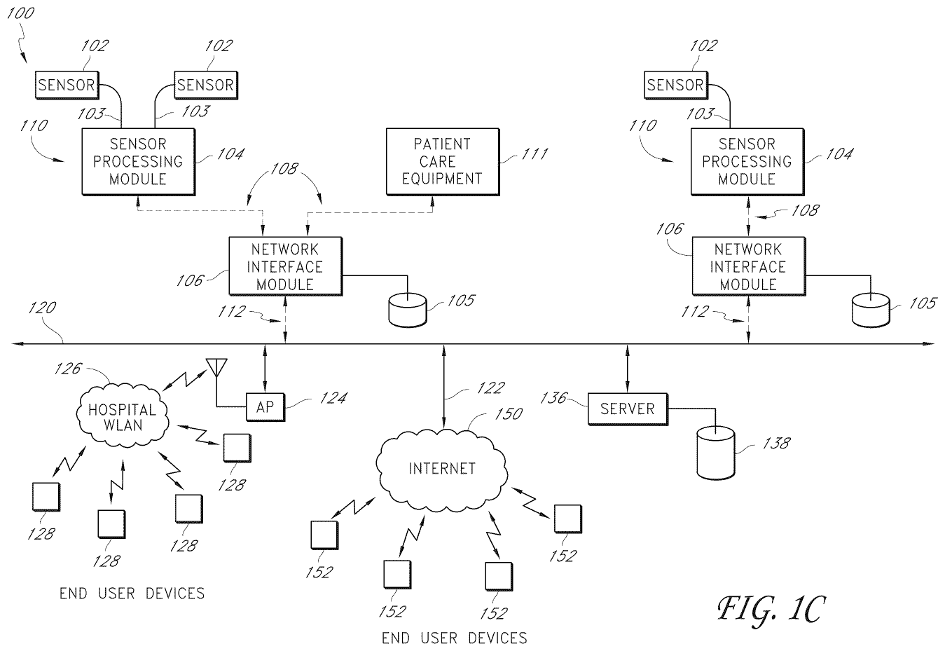

FIG. 1 illustrates a patient monitor 10 connected to a sensor 5 for receiving signals indicative of a physiological condition of a patient. The monitor can include a processor running software configured to process and/or analyze the signal to determine the physiological condition of the patient. The monitor can be running an operating system or other software configured to manage the processing of the signal on the monitor 5 processor. In one embodiment, the sensor 5 comprises a light emitter that emits light of one or more particular wavelengths, or optionally additional emitters for different wavelengths, and one or more optical sensors that detect the light emitted from the emitter or emitters, where the emitted light are reflected by, transflected through, and/or transmitted through tissue of a patient, such as the skin of the patient. The sensor 5 may be configured to transmit raw measurement data to the processor. The sensor 5 and/or the processor may be further configured to perform transformations, analyses, and/or calculations on the transmitted data. This type of sensor is known in the art of pulse oximetry devices and other patient monitor devices, and its implementation is well known to those of ordinary skill in those arts. The sensor may also be an ECG sensor, acoustic sensor, hemoglobin sensor, or other type of physiological sensor. It is contemplated that, in some embodiments, different types of sensors and/or multiple sensors of the same type will be usable within a system.

The monitor 10 includes a third party proprietary software module 12 running on monitor 10. The third party software can run on a dedicated and separate processor to prevent disruption of the primary processor funcionality, or it can operate on the same processor as other physiological parameter processing native to monitor 10. The third party proprietary software can also simply be an add-on to the existing device software. In an embodiment, the third party software is simply front end software, used to configure the display and possibly calculate higher level measurements, whereas the native software operates the backend and low level operational and processing features of a monitoring device.

In an embodiment, patient monitors such as those shown in FIG. 1A can include a virtual machine. The virtual machine 12 can include hardware and/or software. For example it could include one or more software modules running on the monitor's 5 hardware. In an embodiment, the virtual machine and monitor software operate in conjunction with a hypervisor. In an embodiment, the virtual machine runs on the monitor's 5 operating system or a combination of an operating system and hypervisor. Alternatively, the functions described in the present application with respect to a virtual machine can operate on the systems main software platform. For example, in some embodiments, the virtual machine can be an application running on the patient monitor or other patient care device that runs in conjunction with other software running on the patient care device.

In an embodiment, the virtual machine is configured to abstract out and translate measurements, instructions, alarms, management services and other communications into an open architecture specification which is compatible with the third party software 12. This allows software designed to operate on different software platforms to receive information from and communicate efficiently and accurately with the native software on the monitoring device.

For example, in an embodiment, various aspects of the sensor(s) 5, such as, for example, raw data, a sensor measurement, a sensor off alert, an expiration alert, an on/off signal, or the like, is abstracted into a standard format for communication with the third party proprietary software. Various other aspects of the devices can also be abstracted as described in greater detail below.

In an embodiment, the virtual machine is software which emulates the operating system of another device. In an embodiment, the virtual machine can run proprietary software intended to be run on a different proprietary system. For example, in an embodiment, software meant to run on pulse oximeter A will run in the virtual machine on pulse oximeter B. In an embodiment, the virtual machine can run a portion or aspect of the software and is not required to run the entire software. This allows a care provider to pick and choose which portions of each pulse oximeter software to use in a setting without actually using two different devices.

Virtual machines can be adapted to each individual device. Alternatively, one or more virtual machines can be running on a separate device or network location in communication with each patient monitor or patient care device. Thus, also displayed on a selected monitoring device, the third party software can be run remotely from the monitoring device.

In an embodiment, the third party software can be in the form of a down-loadable application designed to operate with the native operating system of the monitoring device, or in conjunction with the virtual machine. For example, various proprietary software packages are capable of determining an indication of the wellness of a patient based on similar measured parameters. By way of example, Masimo Corporation of Irvine CA has developed software referred to as HALO™ which determines a wellness indication of a patient. Other companies have developed competing software packages. In an embodiment, a care giver can choose to run a third party proprietary software application on the monitoring device. In this way, each different care provider can use the same hardware device to pick and choose which software to run. In an embodiment, the application is run on only a portion of the screen so that the rest of the screen is unobstructed and displays the software native to the patient care device. Alternatively a second, third, fourth, etc. application can also be displayed and run simultaneously. In this way, a care provider can use the hardware device and processing developed by one company, but can choose the display and information configuration of another company based on the care giver's preferences.

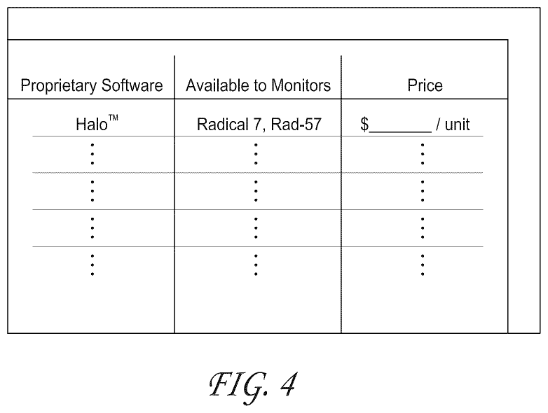

In an embodiment, a care provider can be charged for the hardware separate from the software. For example, a hospital may purchase the hardware separate from the software. In an embodiment, the software can be purchased on a per use or per patient basis. In this way, a hospital will not be charged significant upfront costs for various different software packages that are desired by individual care givers. The decision to use or purchase third party software can be implemented in a home screen on the patient care device. The home screen can provide the user with the option of which preinstalled software the care giver wishes to use as well as the associated pricing. In an embodiment, the patient care device can link to a virtual store (such as the virtual store shown in FIG. 4) accessible via a network 15, such as the Internet or a private hospital network that will allow the care giver to purchase and download desired software onto the device. Alternatively, third party software and applications can be loaded via a portable memory device.

FIG. 1B illustrates a sensor 5 and monitor 10 which communicate with a separate device 11 including the third party software 12 and optionally a virtual machine. Monitor 10 and device 11 communicate through any type of communication methods included on the monitor 10, such as, for example, serial communications, wired or wireless Ethernet, or the like. The third party software communicates with the monitor 10. Alternatively, the monitor 10 emulates the display screen according to the third party software instructions.

In an embodiment that uses a virtual machine, the virtual machine can abstract out device specific information and format the information into a form usable by the third party software. For example, the virtual machine can abstract out the following categories of information: core monitoring services, special services, low level services and content management services. Although described in relation to certain categories, a person of skill in the art will understand from the disclosure herein that other or different categories or category names can be used and the present description is meant by way of example and not limitation.

In some embodiment, certain categories of information are not accessible by the third party software in order to protect functionalities that could disrupt patient monitoring. For example, in an embodiment, low level services and some core monitoring services are not accessible to or capable of being manipulated by the third party software. Rather, the third party software can be limited to receiving information necessary to its functionality and outputting to the monitoring device information for display.

The core monitoring services can include, for example, sensor management, measurement engine management, device management, connectivity management and alarm engine. The sensor management includes services related to sensor channels, channel errors (such as, for example, sensor off, sensor expired, calibration information or the like) and channel exceptions. The measurement engine management includes services related to measurement raw type, measurement limits, alarm levels, alarm priority, numerics including waveforms and measurement numbers, multiple channel i/o, sampling interval, display attributes, multiple levels of alarm thresholds, averaging, 3D alarms, or the like. The device management services include services related to system faults, configurations and settings, accounting, performance, and security (such as, for example, authentication, integrity, privacy and the like). The connectivity management provides services related to interface connections and the alarm engine manages activation and status of alarms.

The special services include location and presence sensing, connectivity management for other local device, such as, for example, bedside integration, integration with body worn devices or sensors, cameras, speakers, video displays, etc. Special services also include power and hosting services and display access services.

The low level services include time services, which, for example, provide a fine grain time and clock sync service. Other low level services include name services and spaces including directories, rules, roles, privileges and scope. The low level services also include a log service. Low level services can include services which are partitioned with higher security levels and limited access rights to prevent tampering or accidental disruptions. For example, the partitions can include protected measurement namespaces that prevent or attempt to prevent one monitoring module from influencing another (e.g., even when both modules use the same naming conventions). Namespaces may be predefined and/or may be generated dynamically. In response to a new measurement module being provided, for instance, a new namespace may be generated for that measurement module. Namespaces can be automatically generated even when modules from different manufacturers are provided.

Content management services include services related to pushing and pulling externally originating content. This can include, such as, for example, messages (text, voice, data, video) both in and out. This allows, for example, advertisements to be displayed or played on a monitoring device. This also allows for direct communications between a care giver at a care center and a patient at a remote (e.g. home) location. For example, a care giver can recommend a treatment to a patient which is displayed on the patient monitor in the patient's home.

In one embodiment, the sensing functions of the monitoring device 110 are decoupled from post-processing functions of the device, so that post-processing functions can be relocated to the third party software. In an embodiment, this relocation feature can be implemented in conjunction with the virtual machine features and emulation features described elsewhere in this specification, and the relocation feature can be implemented using the virtual machine, emulation or an application.

Sensing functions can include receiving data from sensors 102 and/or basic computations or transformations using that received data. Post-processing functions can include computing waveforms using sensor data, calculating averages or other statistics, displaying information about the data on a visual or other display, producing alerts or warnings on the device, or broadcasting data to data monitors such as pagers. Other sensing and post-processing functions are described throughout this specification.

FIG. 1C illustrates an embodiment of a physiological monitoring system 100 including an broader device networking system. This architecture, in various implementations, is a shared, or open, network which includes multiple patient monitoring devices 110, a network bus 120 (e.g., an Ethernet backbone), and a hospital WLAN 126. In addition, the shared network may further include a connection 122 to the Internet 150 or other networks, to end user devices 152 over the Internet 150, and to end user devices 152 over the hospital WLAN 126. The physiological monitoring system 100 of certain embodiments is therefore an enterprise system that achieves a cost-effective replacement for currently available patient monitoring systems.

The physiological monitoring system 100 includes a plurality of bedside devices, e.g., patient monitoring devices 110 and/or patient care equipment 111. The patient monitoring devices 110 of various embodiments include sensors 102, one or more sensor processing modules 104, and a communications module, e.g., network interface module 106. In an embodiment, the network interface module can be built into or form part of the patient monitoring device 110 or patient care equipment 111. In an embodiment, the network interface module is a separate or stand alone piece of hardware which can be configured to communicate with one or more patient monitoring devices 110 or patient care equipment 111. In the depicted embodiment, a patient monitoring devices 110 and patient care equipment 111 are shown. One patient monitoring device includes sensor 102, sensor processing module 104, and network interface module 106. The other patient monitoring device 110 includes two (or more) sensors 102. A person of skill in the art will understand from the present disclosure that any number or combination of sensors, sensor processing modules, or patent care equipment can be used with the presently disclosed system.

In certain embodiments, each patient monitoring device 110 or other patient care equipment are used by one medical patient. The patient monitoring devices 110 and patient care equipment 111 form a network of patient care devices, each of which can communicate with clinicians and other end users over a shared network, including a hospital network 126 and network interfaces to the Internet 150. The network may use standard communications protocols, such as Ethernet, TCP/IP, 802.11b/g/n, IPX/SPX, Appletalk, PPP, and other protocols known to those skilled in the art. As will be understood by a person of skill in the art from the present disclosure, a single piece of patient care equipment or patient monitoring device can also be used by multiple patients or switched between patients.

One or more sensors 102 of the patient monitoring device 110 are attached to a medical patient. These sensors 102 may include ECG sensors, acoustic sensors, pulse oximeters, and other types of sensors. The sensors 102 obtain physiological information from a medical patient and transmit this information to the sensor processing module 104 through cables 103 or through a wireless connection (not shown). In certain embodiments, the physiological information includes one or more physiological parameters or values and waveforms corresponding to the physiological parameters.

The sensor processing module 104 receives physiological information from the sensors 102. The sensor processing module 104 of certain embodiments includes a circuit having a processor, input ports for receiving the physiological information, software for processing the physiological information in the processor, an optional display, and optionally an input device (e.g., a keyboard). In addition, the sensor processing module 104 contains one or more output ports, such as serial ports. For example, an RS232, RS423, or autobaud RS232 (serial interface standard) port or a universal serial bus (USB) port may be included in the sensor processing module 104. Patient care equipment 111 can likewise include input and output interfaces for receiving and transmitting communications and instructions.

In certain embodiments, the sensor processing module 104 generates waveforms from signals received from the sensors 102. The sensor processing module 104 may also analyze single or multiparameter trends to provide early warning alerts to clinicians prior to an alarm event. In addition, the sensor processing module 104 in certain embodiments generates alarms, otherwise known as faults, failures, or alerts, in response to physiological parameters exceeding certain safe thresholds.

Example alerts include no communication with pulse oximeter, alarm silenced on pulse oximeter, instrument low battery (pulse oximeter), and transmitter low battery. Example alarms include SpO₂ levels and alarms, high and low SpO₂, high and low PR, HbCO level and alarms, HbMET level and alarms, pulse rate and alarms, no sensor, sensor off patient, sensor error, low perfusion index, low signal quality, HbCO, HbMET, PI trend alarm, and desat index alarm.

The network interface module 106 in the depicted embodiment is connected to one or more sensor processing modules 104 or patient care equipment 111 through one or more connectors 108, which may be serial connectors corresponding to the serial ports in the sensor processing modules 104. Alternatively, the connectors 108 may be any hard wired or wireless communications types including wired or wireless Ethernet, telephone lines, Wi-Fi, etc. Dashed lines on the connector 108 indicate that the network interface module 106 of certain embodiments is not permanently attached to the sensor processing modules 104. In alternative embodiments (not shown), however, the network interface module 106 is contained within a sensor processing module 104 or patient care equipment 111.

The network interface module 106 in various implementations includes a processor, an input port (such as a standard RS232 serial port, Ethernet port, wireless transceiver, etc.), a network output port such as an Ethernet port, Ethernet transceiver serial interface, etc., and software which enables the network interface module 106 to act as a network-communications enabled device. In addition, the network interface module 106 includes a storage device 114, which may be included within the network interface module 106 or attached separately to the network interface module 106.

The network interface module 106 manages the connectivity overhead for initiating and maintaining connectivity with end user devices over the shared network. In certain embodiments, the network interface module 106 manages connectivity by acting as a microserver or web server. In such instances, the network interface module 106 is a network connection enabled device. As a web server, the network interface module 106 establishes direct connections to the Internet 150, such that an end user may access web pages or third party software stored on the storage device 105 of the network interface module 106 or access the Internet through the network interface module 106. In an embodiment, the network interface module 106 therefore does not require a separate server for connecting to the Internet 150. In one embodiment, the network interface module 106 connects to the Internet 150 directly through a modem, such that the connection 122 includes a modem. In managing connectivity over the shared network, the network interface module 106 may also perform security management functions, such as user authentication.

In certain embodiments, the network interface module 106 sends data over the shared network through an access point 124 or other wireless or wired transmitter. Alternatively, the network interface module 106 may communicate directly with end users over the Internet 150. End users such as clinicians carrying notifier devices, e.g., end user devices 128, 152 connected to the hospital WLAN 126 may receive real-time viewing of physiological patient parameters and waveforms on demand or in the event of an alarm or alert. End users can also provide instructions or other communications to the patient monitor 110 or patient care equipment 111 using the same end user devices. Real-time or slightly delayed transmission of physiological information in certain embodiments comports with standards for alarm latency in compliance with Joint Commission on Accreditation of Healthcare Organizations (JCAHO) standards for effective alarm response. The network interface module 106 of certain embodiments therefore adds functionality equivalent to a central nurses' station. These communications between network components can include information generated by third party software running on the individual monitoring devices.

In certain embodiments, the network interface module 106 performs context management. In one embodiment, context management includes associating context information with physiological information to form a contextual data package. Context information may include several categories of information, including the categories of context information related to the network interface module 106, context information related to the medical patient, context information related to usage of the network interface module 106, and context information related to a network connection. Within one or more of these context categories, context information might include a patient name, a patients' unique hospital identification number, patient location, an identification number for a network interface module 106, time stamps for events occurring in the physiological monitoring system 100, environmental conditions such as changes to the state of the network and usage statistics of the network interface module 106, and identification information corresponding to the network link (e.g., whether the network connection is WiFi or Ethernet). In one embodiment, the context information in the contextual data package may include all of or any subset of context information from one or more of the context categories. In an embodiment, the context information and measurement information are packaged by the virtual machine.

In another embodiment (not shown), end user devices 128, 152 include one way POCSAG Pagers having a 2 line display with audible and vibrate mode, of suitable size and durability for severe mechanical environments typical of hospital general floor settings. In yet another embodiment, the end user devices 128, 152 include two way paging systems, such as Motorola Flex and WLAN pagers. One advantage of two-way paging is the ability to confirm message receipt and the ability to remotely silence alarms. Wireless PDAs may also be used by end users based on ruggedness and acceptable form factors as determined by an end user. An example of such a device is the Symbol Technology MC50 PDA/Barcode Scanner.

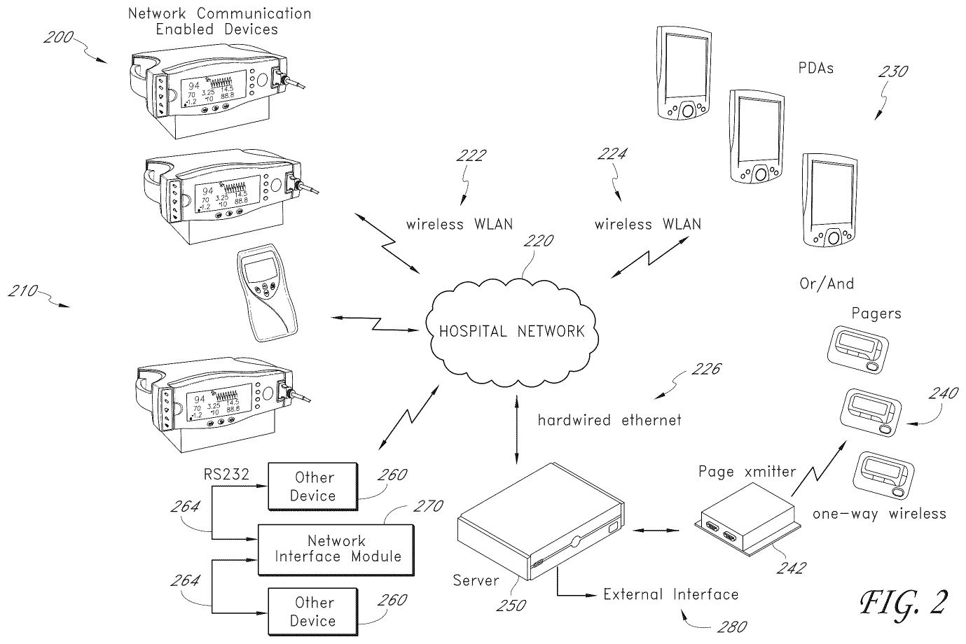

FIG. 2 depicts another embodiment of a physiological monitoring system 200 of the present disclosure. The physiological monitoring system 200 (or alternatively other patient care equipment, not shown in FIG. 2) includes network communications enabled devices 210. The network communications enabled devices 210 are connected directly to a hospital network 220 through a wireless connection. In certain embodiments, the network communications enabled devices 210 include sensors and sensor processing modules, similar to the sensors 102 and sensor processing modules 104 of FIG. 1. Certain of these network communications enabled devices 210 are bedside devices, and others are handheld or otherwise patient-worn devices that may be used by an ambulatory (mobile) patient.

The hospital network 220 transmits physiological information and context information to clinician notifier devices, including pagers 240, PDAs 230 as well as cell phones, portable and stationary computers or any other communications devices. This information can include information generated by the third party software. In certain embodiments, the hospital network 220 utilizes a server 250 to transmit contextual data packages to a page transmitter 242, which further transmits the data to one-way wireless pagers 240. An external interface 280 may be coupled with the server 250. The external interface 280 could include one or more of the following: enterprise paging, nurse call systems, wide area paging systems, enterprise clinical and patient information systems, and third party monitoring and surveillance systems.

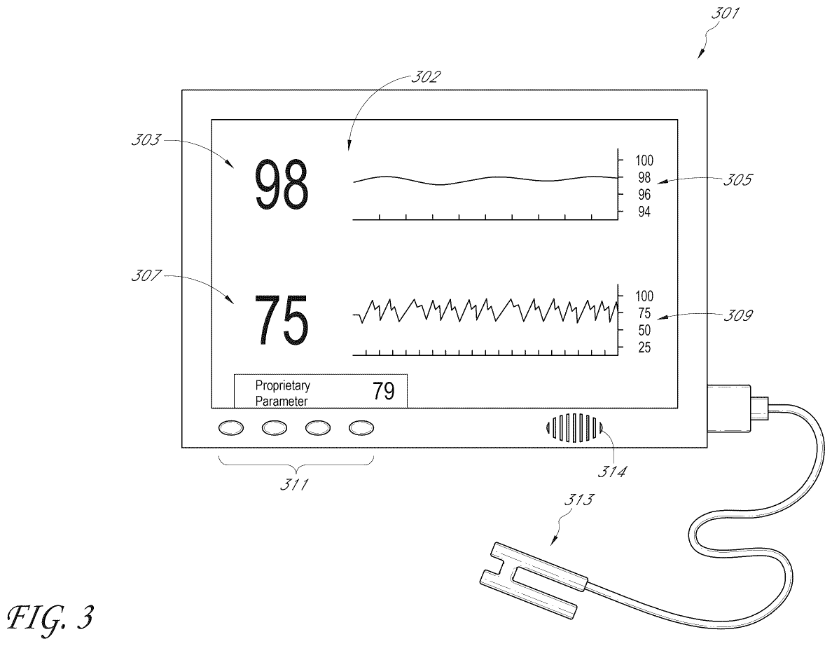

FIG. 3 illustrates an embodiment of a pulse oximeter 301. The pulse oximeter 301 includes a display screen 302 which displays various parameters 303 and 307 along with parameter trends 305 and 309. The pulse oximeter includes a speaker 314, inputs 311 and sensor 313. In an embodiment, proprietary parameters can be included using third party proprietary software as described above.

FIG. 4 illustrates an embodiment of a software package selection screen. This display screen can be internal to a monitoring device or can be accessible via a network, such as a private hospital network or the Internet. In an embodiment, the display screen illustrates the available proprietary software, along with the price. The price can be based on a per software activation or download or it can be based on a per patient use.

Those of skill in the art will understand that information and signals can be represented using any of a variety of different technologies and techniques. For example, data, instructions, commands, information, signals, bits, symbols, and chips that can be referenced throughout the above description can be represented by voltages, currents, electromagnetic waves, magnetic fields or particles, optical fields or particles, or any combination thereof.

Those of skill will further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the present disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, conventional processor, controller, microcontroller, state machine, etc. A processor can also be implemented as a combination of computing devices, e.g., a combination of a DSP and a microprocessor, a plurality of microprocessors, one or more microprocessors in conjunction with a DSP core, or any other such configuration. In addition, the term “processing” is a broad term meant to encompass several meanings including, for example, implementing program code, executing instructions, manipulating signals, filtering, performing arithmetic operations, and the like.

The steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, a DVD, or any other form of storage medium known in the art. A storage medium is coupled to the processor such that the processor can read information from, and write information to, the storage medium. In the alternative, the storage medium may be integral to the processor. The processor and the storage medium can reside in an ASIC. The ASIC can reside in a user terminal. In the alternative, the processor and the storage medium can reside as discrete components in a user terminal.

The modules can include, but are not limited to, any of the following: software or hardware components such as software object-oriented software components, class components and task components, processes, methods, functions, attributes, procedures, subroutines, segments of program code, drivers, firmware, microcode, circuitry, data, databases, data structures, tables, arrays, or variables.

In addition, although this disclosure has been disclosed in the context of a certain preferred embodiment, it will be understood by those skilled in the art that the present disclosure extends beyond the specifically disclosed embodiment to other alternative embodiments and/or uses of the disclosure and obvious modifications and equivalents thereof. In particular, while the present system and methods have been described in the context of a particularly preferred embodiment, the skilled artisan will appreciate, in view of the present disclosure, that certain advantages, features and aspects of the system, device, and method may be realized in a variety of other applications and software systems. Additionally, it is contemplated that various aspects and features of the disclosure described can be practiced separately, combined together, or substituted for one another, and that a variety of combination and subcombinations of the features and aspects can be made and still fall within the scope of the disclosure. Furthermore, the systems described above need not include all of the modules and functions described in the preferred embodiments. Thus, it is intended that the scope of the present disclosure herein disclosed should not be limited by the particular disclosed embodiment described above, but should be determined only by a fair reading of the claims that follow.

1.-6. (canceled)

7. A method for displaying a health parameter of a user, the method comprising:
determining, with a first software, one or more physiological measurements from a measured raw data acquired by a sensor;
downloading a second software, wherein the second software is configured to execute one or more post-processing functions on the determined one or more physiological measurements;
providing, with the first software, an option in a first graphical user interface presented on a display for a user to select a determination of a health parameter that is specific to the second software;
selecting the second software for the determination of the health parameter;
receiving an indication that the second software was selected from the option in the graphical user interface;
enabling the second software to execute the one or more post-processing functions on the determined one or more physiological parameters based on the selection of the second software, wherein the one or more post-processing functions are configured to determine the health parameter; and
displaying, on a second graphical user interface, the health parameter on a display along with the one or more physiological parameters determined by the first software, wherein the second graphical user interface is generated by the first software.

8. The method of claim 7, further comprising generating a format for the determined one or more physiological parameters to enable the one or more post-processing functions by the second software.

9. The method of claim 7, wherein the determination of the one or more physiological measurements is separate from the post-processing functions.

10. The method of claim 7, wherein the post-processing functions include producing alerts on the display.

11. The method of claim 7, wherein the health parameter is proprietary to the second software.

12. The method of claim 7, further comprising displaying the health parameter as determined by the second software and the one or more physiological measurements as determined by the first software simultaneously on the display.

13. The method of claim 7, further comprising running the second software simultaneously with the first software.

14. The method of claim 7, wherein the second software is configured to operate with the first software or in conjunction with a virtual machine.

15. The method of claim 7, wherein the health parameter comprises a wellness indicator specific to the second software.

16. A system for displaying a health parameter of a user, the system comprising one or more hardware processors configured to:
determine one or more physiological measurements from measured raw data acquired by a sensor with a first software;
download a second software, wherein the second software is configured to execute one or more post-processing functions on the determined one or more physiological measurements;
provide, with the first software, an option in a first graphical user interface presented on a display for a user to select a determination of a health parameter that is specific to the second software;
select the second software for the determination of the health parameter;
receive an indication that the second software was selected from the option in the graphical user interface;
enable the second software to execute the one or more post-processing functions on the determined one or more physiological parameters based on the selection of the second software, wherein the one or more post-processing functions are configured to determine the health parameter; and
display, on a second graphical user interface, the health parameter on a display along with the one or more physiological parameters determined by the first software, wherein the second graphical user interface is generated by the first software.

17. The system of claim 16, wherein the one or more hardware processors are further configured to generate a format for the determined one or more physiological parameters to enable the one or more post-processing functions by the second software.

18. The system of claim 16, wherein the determination of the one or more physiological measurements is separate from the post-processing functions.

19. The system of claim 16, wherein the post-processing functions include producing alerts on the display.

20. The system of claim 16, wherein the health parameter is proprietary to the second software.

21. The system of claim 16, wherein the one or more hardware processors are further configured to display the health parameter as determined by the second software and the one or more physiological measurements as determined by the first software simultaneously on the display.

22. The system of claim 16, wherein the one or more hardware processors are further configured to run the second software simultaneously with the first software.

23. The system of claim 16, wherein the second software is configured to operate with the first software or in conjunction with a virtual machine.

24. The system of claim 16, wherein the health parameter comprises a wellness indicator specific to the second software.
